(19) United States
(12) Patent Application Publication
(10) Pub. No.: US 2014/0166035 A1
(43) Pub. Date: Jun. 19, 2014

(54) TWO PHASE DEVELOPER FOR MEDIUM OXIDATIVE COLOR CHANGE

(71) Applicant: Henkel AG & Co. KGaA, Duesseldorf (DE)

(72) Inventors: Sabine Babiel, Moers (DE); Anja Reichert, Duesseldorf (DE); Carsten Brake, Essen (DE); Mechtild Grunwald, Langenfeld (DE); Barbara Augustin-Castro, Vaals (NL)

(73) Assignee: Henkel AG & Co. KGaA, Duesseldorf (DE)

(21) Appl. No.: 14/108,744

(22) Filed: Dec. 17, 2013

(30) Foreign Application Priority Data
Dec. 18, 2012 (DE) 10 2012 223 564.7

(51) Int. Cl. A61K 8/37; A61Q 5/10; A61K 8/92; A61K 8/81; A61K 8/22; A61K 8/46

(52) U.S. Cl. CPC A61K 8/37; A61K 8/22; A61K 8/463; A61K 8/922; A61K 8/8152; A61Q 5/10
USPC 132/208; 424/70.1; 424/70.21; 424/70.24

(57) ABSTRACT

The subject matter of the present Application is cosmetic agents for treating keratinic fibers, which encompass at least two phases present alongside one another but separated from one another by a phase boundary, where the first phase (I) represents an aqueous phase that contains at least one chemical oxidizing agent, and where the second phase (II) represents a hydrophobic oil phase that contains a mixture of isopropyl palmitate (II-1) and at least one further liquid, branched carboxylic acid ester (II-2). The compositions are particularly shelf-stable and cold-stable, and are notable for outstanding care-providing properties.

### CROSS REFERENCE TO RELATED APPLICATION

This application claims priority to German Patent Application No. DE 10 2012 223 564.7, filed Dec. 18, 2012, its contents hereby incorporated in its entirety.

### TECHNICAL FIELD

The subject matter of the present disclosure is a cosmetic agent for treating keratinic fibers that is notable for two phases separated from one another, one of the phases representing an aqueous phase and the other phase representing a hydrophobic oil phase. These agents contain at least one chemical oxidizing agent as well as a mixture of isopropyl palmitate and a further liquid, branched carboxylic acid ester. In terms of their property as oxidizing preparations, these agents are used as a developer preparation for oxidation coloring processes or lightening agents. A further subject of the present disclosure is therefore a method for changing the color of keratinic fibers in which the agents according to the present invention are applied onto keratinic fibers.

### BACKGROUND

One skilled in the art knows of a variety of coloring systems, depending on the requirements for the coloring process, for making available color-changing cosmetic agents, in particular for the skin or for keratin-containing fibers such as e.g. human hair. For permanent, intense color having corresponding fastness properties, so-called “oxidizing” coloring agents are used. Such coloring agents usually contain oxidation dye precursors called “developer components” and “coupler components” that, under the influence of oxidizing agents or atmospheric oxygen, form the actual dyes with one another. Oxidizing coloring agents re notable for outstanding, long-lasting color results. For temporary coloring, it is usual to use coloring or toning agents that contain so-called “substantive” dyes as a coloring component. In addition to coloring, lightening of one's own hair color, resp. hair-bleaching, is a very special desire of many consumers, since a blond hair color is regarded as attractive and as desirable in terms of fashion. When substrates need to be lightened or in fact bleached, the dyes coloring the substrate are usually decolorized oxidatively using corresponding oxidizing agents, such as hydrogen peroxide.

In order to yield optimum coloring performance, oxidative coloring agents generally require an alkaline pH for color development, in particular between pH 9.0 and pH 10.5. In addition, the application time for corresponding color results is usually between 10 and 45 min. It is therefore necessary for the ready-to-use coloring agent to be formulated and packaged in such a way that the coloring agent on the one hand can be effectively distributed onto the keratinic fibers to be colored, but on the other hand remains in the fibers to be colored during the application time. It is advantageous for this if the coloring agent has a specific viscosity that enables application of the agent but also allows the agent to remain where it has been applied. This viscosity can be adjusted in the ready-to-use coloring agent using polymeric thickening agents; this thickening agent can be contained both in the color-changing preparation or in the oxidizing agent preparation.

In order to enable good mixing of a color-changing preparation and oxidizing agent preparation, it is advantageous if the color-changing preparation and oxidizing agent preparation exhibit good flowability.

In addition, the oxidizing agent preparations are also intended to possess care-providing properties, and to express them by way of a suitable visual, cosmetic appearance of the hair. Two-phase oxidizing agent preparations that contain, besides an aqueous phase containing oxidizing agent, a further care-providing hydrophobic phase, are suitable in particular for this.

Two-phase oxidizing agent preparations having care-providing properties are known from DE 10 2010 003264 A1. This describes two phases that are packaged in contact with one another, one phase representing an aqueous oxidizing agent phase and a further phase representing a hydrophobic oil phase.

It has been found that the shelf stability of the compositions according to DE 10 2010 003264 A1 is not always satisfactory. For example, it has been found on occasion that the flowability of the hydrophobic oil phase can change in the context of temperature fluctuations (in particular in the range of colder temperatures), which negatively affects good miscibility of the color-changing preparation and the oxidizing agent preparation.

The object of the present invention is therefore to make available a cosmetic agent with which the aforementioned disadvantages can be reduced. It is the object of the present invention in particular to make available a two-phase oxidation preparation for oxidative color-changing agents of keratinic fibers, the aqueous phase and hydrophobic oil phase of which can be mixed with one another without difficulty even after extended storage and/or in a context of temperature fluctuations (in particular at temperatures below 15° C.).

In particular, the flowability of the hydrophobic phase is intended not to be degraded even upon storage at lower temperatures.

A further object of the invention is to improve the care-providing properties of two-phase oxidation preparations. “Improved care-providing properties” are to be understood, for example, as improved wet and dry combability and/or greater shine for the treated hair, and/or the achievement of more uniform, more homogeneous coloring results. Furthermore, other desirable features and characteristics of the present invention will become apparent from the subsequent detailed description of the invention and the appended claims, taken in conjunction with the accompanying drawings and this background of the invention.

### DETAILED DESCRIPTION

It has now been found, unforeseeably, that these objects can be achieved in particular fashion by means of special two-phase oxidative preparations that contain, besides an oxidizing agent, a mixture of isopropyl palmitate and at least one further liquid hydrophobic oil.

A first subject of the present invention is therefore a cosmetic agent for treating keratinic fibers which encompasses at least two phases present alongside one another but separated from one another by a phase boundary, where the first phase (I) represents an aqueous phase that contains at least one chemical oxidizing agent, and where the second phase (II) represents a hydrophobic oil phase that contains a mixture of isopropyl palmitate (II-1) and at least one further liquid, branched carboxylic acid ester (II-2).

“Keratin-containing” resp. “keratinic” fibers are understood according to the present invention as furs, wool, feathers, and in particular human hair. Although the use according to the present invention is suitable principally for coloring and/or lightening keratin-containing fibers, nothing in principle conflicts with use in other sectors as well.

A crucial feature of the preparation according to the present invention is its two-phase nature, the two phases not being miscible with one another and the two phases being present in two layers one above another, with direct contact via a common interface.

In the preparation according to the present invention, phase (I) is preferably present in at least the same weight proportion as phase (II). Phase (I) is preferably present in excess. The weight ratio of phase (I) to phase (II) preferably has a value from about 99 to about 1 to about 50 to about 50, preferably from about 98 to about 2 to about 70 to about 30, particularly preferably from about 95 to about 5 to about 80 to about 20.

An obligatory feature of the present invention is phase (I), which is aqueous. This is understood to mean that phase (I) is an aqueous composition that can contain about 3 to about 70 wt % of a C₁ to C₄ alcohol, based on the total weight of the utilization mixture. Preferred C₁ to C₄ alcohols are in particular ethanol resp. isopropanol.

In a preferred embodiment the cosmetic agent contains at least about 30 wt %, in particular at least about 50 wt %, water, based on the total weight of the agent.

The first phase (I) further contains at least one chemical oxidizing agent. The term “chemical oxidizing agent” is intended to make it clear that this is a supplementary, added oxidizing agent and not, for example, an oxidizing agent present in the environment, e.g. atmospheric oxygen. Hydrogen peroxide is preferably used as an oxidizing agent according to the present invention. Hydrogen peroxide is used either as a preferably aqueous solution, or in the form of a solid addition compound of hydrogen peroxide with inorganic or organic compounds such as, for example, sodium perborate, sodium percarbonate, magnesium percarbonate, sodium percarbamide, polyvinylpyrrolidinone.nH₂O (where n is a positive number greater than 0), urea peroxide, and melamine peroxide. Aqueous phases (I) preferred according to the present invention contain aqueous hydrogen peroxide solutions. The concentration of a hydrogen peroxide solution is determined on the one hand by regulatory provisions and on the other hand by the desired effect. By preference, about 3-wt % to about 12-wt % solutions of hydrogen peroxide in water are used.

An embodiment of the first subject of the invention is therefore characterized in that the chemical oxidizing agent of phase (I) is selected from hydrogen peroxide and/or a solid addition product thereof with inorganic and/or organic compounds.

The preparations according to the present invention contain, with particular preference, hydrogen peroxide. Agents according to the present invention for changing the color of keratinic fibers that are particularly preferred here contain about 0.5 to about 18 wt %, by preference about 1 to about 15 wt %, particularly preferably about 2.5 to about 12 wt %, and in particular about 3 to about 9 wt % hydrogen peroxide (calculated as 100% H₂O₂.

In accordance with the present invention, the second phase (II) is hydrophobic in nature. The hydrophobic phase (II) according to the present invention is not miscible with the aqueous phase (I) containing the oxidizing agent. Hydrophobic phases—also called “lipophilic” phases—contain fatty substances that usually contain nonpolar organic compounds such as hydrocarbon compounds, long-chain triglycerides, silicone oils, esters, or ethers, as well as perhalogenated compounds.

The hydrophobic oil phase (II) of the present invention is notable for the fact that it contains a mixture of isopropyl palmitate (II-1) and at least one further liquid, branched carboxylic acid ester (II-2). The term “liquid” refers here to carboxylic acid esters that are liquid at room temperature and under standard pressure. Carboxylic acid esters suitable according to the present invention are those that possess no or only very little water solubility, i.e. a water solubility of less than about 1 g per 1 L of water under standard conditions.

It has been found that the low-temperature stability of the agents according to the present invention is particularly pronounced when isopropyl palmitate (II-1) is present at a specific weight ratio in terms of the sum of all further carboxylic acid esters (II-2) in phase (II).

In a further preferred embodiment, the weight ratio of isopropyl palmitate (II-1) to the sum of all further carboxylic acid esters (II-2) in phase (II) is therefore about 20:80 to about 80:20, preferably about 30:70 to about 70:30, more preferably about 40:60 to about 60:40, and particularly preferably about 45:55 to about 55:45.

Suitable “liquid, branched carboxylic acid esters” are preferably understood as esters of straight-chain or branched C₂ to C₃₀ carboxylic acids with branched C₃ to C₃₀ alcohols, where the term “carboxylic acids” is understood to mean both mono- and dicarboxylic acids. Monocarboxylic acid esters are preferred.

Examples of C₃ to C₃₀ alcohols suitable according to the present invention are isopropanol, isobutanol, 2-methyl-2-butanol, 2-methyl-3-butanol, 2-methyl-2-pentanol, 2-methyl-3-pentanol, 2-methyl-4-pentanol, 2-ethylhexanol, 2-ethylheptanol, isooctanol, isononanol, isodecyl alcohol, isotridecanol, hexyldecyl alcohol, octyldodecanol, and/or triisodecanol.

Examples of carboxylic acids suitable according to the present invention are acetic acid, propanoic acid, octanoic acid, neooctanoic acid, neopentanoic acid, nonanoic acid, isononanoic acid, decylic acid, neodecanoic acid, lauric acid, isolauric acid, myristic acid, isomyristic acid, coconut acid, palmitic acid, isopalmitic acid, stearic acid, isostearic acid and/or oleic acid.

Particularly preferred liquid, branched carboxylic acid esters that are suitable for mixing with isopropyl palmitate in phase (II) are selected from isopropyl isostearate, 2-ethylhexyl palmitate, hexyldecyl palmitate, hexyldecyl stearate, isopropyl stearate, hexyldecyl laurate, isodecyl neopentanoate, isononyl isononanoate, 2-ethylhexyl stearate, isopropyl oleate, isooctyl stearate, isononyl stearate, isocetyl stearate, isononyl isononanoate, isotridecyl isononanoate, cetearyl isononanoate, propylheptyl caprylate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, and/or diisotridecyl acetate.

Particularly shelf-stable and cold-stable agents according to the present invention can be obtained when at least one ester that has a cloud point<7° C. is used as a further liquid, branched carboxylic acid ester.

In a further preferred embodiment, the isopropyl palmitate of phase (II) is therefore mixed with at least one of the esters recited previously that has a cloud point preferably <7° C., more preferably <5° C., and in particular <3° C.

In a first particularly preferred embodiment, phase (II) comprises a mixture of isopropyl palmitate and isopropyl isostearate.

In a second particularly preferred embodiment, phase (II) comprises a mixture of isopropyl palmitate and 2-ethylhexyl palmitate (Octyl Palmitate).

In a third particularly preferred embodiment, phase (II) comprises a mixture of isopropyl palmitate and hexyldecyl palmitate.

In a fourth particularly preferred embodiment, phase (II) comprises a mixture of isopropyl palmitate and hexyldecyl stearate.

For stability reasons, it is essential that the two phases (I) and (II) be present in a manner that is clearly and sharply separated from one another, and that no intermediate phases or mixed phases occur in the phase boundary region. Clouding of the phases due to streaking or partial emulsion regions in the phases are also to be avoided. It is therefore particularly advantageous if the hydrophobic oil phase in particular is present in clear and transparent fashion. “Clear” and “transparent” are to be understood here as a transmittance of at least 60%, preferably at least 80%. This transmittance is measured by UV/VIS spectrometry. The transmittance can be measured at different wavelengths, preferably at room temperature, at a wavelength λ=600 nm and with a cuvette length of about 10 mm.

The agent of the first subject of the invention preferably contains about 3 to about 30 wt %, more preferably about 4 to about 25 wt %, particularly preferably about 5 to about 20 wt %, and in particular about 7.5 to about 15 wt % hydrophobic oil phase (II).

It can optionally contain, besides the mixture of isopropyl palmitate and the at least one liquid, branched carboxylic acid ester, further oil components such as e.g. hydrocarbon compounds, long-chain triglycerides, silicone oils, esters, or ethers, as well as perhalogenated compounds.

An objective of the present invention was to manufacture two-phase agents in which both phases were intended to exhibit sufficient flowability that good blending of the phases can be achieved immediately before utilization. Optimally, however, the viscosity of the agent should rise after blending of the two phases, so that the agent can be more easily applied onto the hair fibers and remains there for the utilization time without running off.

It has proven advantageous for this purpose to add to the aqueous phase, besides the chemical oxidizing agent, at least one suitable polymeric thickening agent.

In a further embodiment, particularly preferred agents according to the present invention are therefore characterized in that they furthermore contain at least one anionic polymeric thickening agent in the aqueous phase (II). Preferred anionic polymeric thickening agents are selected from crosslinked or uncrosslinked copolymers that contain at least two different monomers from the group of acrylic acid, methacrylic acid, C₁ to C₆ alkyl esters of acrylic acid, and/or C₁ to C₆ alkyl esters of methacrylic acid.

Particularly preferred anionic copolymers are, for example, copolymers of acrylic acid, methacrylic acid, or C₁ to C₆ alkyl esters thereof, as marketed under the INCI name Acrylates Copolymers. The combination of methacrylic acid and ethyl acrylate, as well as optionally crosslinking multifunctional monomers, is preferred in this context. A preferred commercial product for this is, for example, Aculyn® 33 resp. 33A that is offered by the Rohm & Haas company.

The anionic polymeric thickening agents can be used preferably in a total quantity from about 0.1 to about 15 wt %, more preferably from about 1 to about 10 wt %, and in particular from about 1.5 to about 7.5 wt %, where the quantities refer to the total weight of the agent according to the present invention.

In order to improve the separation of hydrophilic phase (I) and hydrophobic phase (II) and to obtain a clear hydrophobic phase (II), the tendency of the agent to form a stable emulsion must be reduced. It is therefore preferred according to the present invention if the agent contains only a small proportion of surface-active substances. “Surface-active substances” for purposes of the invention are considered to be emulsifier agents and surfactants. Surface-active substances are notable for hydrophobic and hydrophilic structural features, and thus enable blending of the phases accompanied by the formation of micelles and stable emulsions. Because the present invention explicitly does not encompass any emulsions, but instead contains two phases that are present separately from one another, it has proven to be particularly advantageous according to the present invention that the agent contains nonionic, anionic, zwitterionic, and/or amphoteric surfactants and/or emulsifier agents at a total weight of less than 5 wt %, more preferably less than 3 wt %, more preferably less than 1 wt %, extraordinarily preferred at a total weight of about 0.05 to about 0.5 wt %, more preferably about 0.1 to about 0.3 wt %, based in each case on the total weight of the agent according to the present invention.

“Anionic surfactants” for purposes of the invention are all anionic surface-active substances suitable for use on the human body. These are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of such anionic surfactants are, in each case in the form of the sodium, potassium, and ammonium and mono-, di, and trialkanolammonium salts having 2 to 4 carbon atoms in the alkanol group: linear and branched fatty acids having 8 to 30 carbon atoms (soaps); ethercarboxylic acids, in particular of the formula RO(CH₂CH₂O)ₓCH₂COOH, in which R is a linear alkyl group having 8 to 30 carbon atoms and x=0 or is 1 to 16; acyl sarcosides; acyl taurides; acyl isethionates; sulfosuccinic acid mono- and dialkyl esters and sulfosuccinic acid monoalkylpolyoxyethyl esters; linear alkanesulfonates; linear α-olefinsulfonates; sulfonates of unsaturated fatty acids; α-sulfo fatty acid methyl esters of fatty acids; alkyl sulfates and alkyl ether sulfates, in particular of the formula RO(CH₂CH₂O)ₓSO₃H in which R denotes a linear alkyl group having 8 to 30 carbon atoms and x denotes zero or a number from 1 to 12; mixtures of surface-active hydroxysulfonates; sulfated hydroxyalkylpolyethylene glycol ethers and/or hydroxyalkylenepropylene glycol ethers; esters of tartaric acid and citric acid with alcohols; alkyl and/or alkenyl ether phosphates of the formula RO(C₂H₄O)ₓP(═O)(OH)(OR′) in which R denotes an aliphatic, optionally unsaturated hydrocarbon residue having 8 to 30 carbon atoms, R′ denotes hydrogen, a (CH₂CH₂O)_{y}R residue, and x and y mutually independently denote a number from 1 to 10; sulfated fatty acid alkylene glycol esters of the formula RC(O)O(alkO)ₙSO₃H, in which R denotes a linear or branched, aliphatic, saturated and/or unsaturated alkyl residue having 6 to 22 carbon atoms, alk denotes CH₂CH₂, CHCH₃CH₂, and/or CH₂CHCH₃, and n denotes a number from 0.5 to 5; as well as monoglyceride sulfates and monoglyceride ether sulfates.

“Zwitterionic surfactants” refers to those surface-active compounds that carry in the molecule at least one quaternary ammonium group and at least one carboxylate, sulfonate, or sulfate group. Examples of such zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Amphoteric surfactants” are understood to be those surface-active compounds that contain in the molecule, in addition to a C₈ to C₂₄ alkyl or acyl group, at least one free amino group and at least one —COOH or —SO₃H group, and are capable of forming internal salts. Usual amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Examples of amphoteric surfactants are N-cocalkylaminopropionate, cocacylaminoethylaminopropionate, and C₁₂ to C₁₈ acyl sarcosine.

Nonionic surfactants and emulsifier agents contain as a hydrophilic group, for example, a polyol group, a polyalkylene glycol ether group, or a combination of a polyol glycol ether and polyglycol ether group. Such compounds are, for example: addition products of about 1 to about 50 mol ethylene oxide and/or 0 to about 5 mol propylene oxide with linear and branched fatty alcohols having 8 to 30 carbon atoms, with fatty acids having 8 to 30 carbon atoms, and with alkylphenols having 8 to 15 carbon atoms in the alkyl group; addition products, end-capped with a methyl or C₂ to C₆ alkyl residue, of about 1 to about 50 mol ethylene oxide and/or 0 to about 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydrol LS, Dehydrol LT (BASF); polyglycerol esters and alkoxylated polyglycerol esters, for example poly(3)glycerol diisostearate (commercial product: Lameform TGI (Henkel)) and poly(2)glycerol polyhydroxystearate (commercial product: Dehymuls PGPH (Henkel)); polyol fatty acid esters, for example the commercial product Hydagen HSP (BASF) or Sovermol grades (BASF); more highly alkoxylated, propoxylated, and in particular ethoxylated mono-, di-, and triglycerides having a degree of alkoxylation greater than 5, for example glycerol monolaurate+20 ethylene oxide and glycerol monostearate+20 ethylene oxide; amine oxides; hydroxy mixed ethers; sorbitan fatty acid esters and addition products of ethylene oxide with sorbitan fatty acid esters, for example the polysorbates and sorbitan monolaurate+20 mol ethylene oxide (EO); sugar fatty acid esters and addition products of ethylene oxide with sugar fatty acid esters; addition products of ethylene oxide with fatty acid alkanolamides and fatty amines; fatty acid N-alkylglucamides; alkylphenols and alkylphenol alkoxylates having 6 to 21, in particular 6 to 15 carbon atoms in the alkyl chain and 5 to 30 ethylene oxide and/or propylene oxide units; alkylpolyglycosides corresponding to the general formula RO—(Z)ₓ, where R denotes alkyl, Z sugar, and x the number of sugar units.

Further included among the nonionic emulsifier agents for purposes of the invention are the polymerization products of ethylene oxide and propylene oxide with saturated or unsaturated alcohols; alkyl esters of saturated or unsaturated fatty acids or alkylphenols, and alkoxylates thereof; in particular, ethylene glycol ethers of fatty alcohols; mixed ethylene and propylene glycol ethers with fatty alcohols; fatty acid esters of sorbitan and polyethylene glycol; esters of non-hydroxylated C₆ to C₃₀ alkylmonocarboxylic acids with polyethylene glycol; and addition products of alkyl phenols with ethylene oxide and/or propylene oxide.

It may further be advantageous, in order to separate the hydrophilic and hydrophobic phase in the agent according to the present invention, if electrolytes are additionally added to the agent. “Electrolytes” are usually understood as charged ionic inorganic and organic compounds that contain only a very slightly expressed hydrophobic component, or none at all. Preferred electrolytes are readily water-soluble salts, in particular alkali metal and alkaline earth metal salts of mineral acids and organic acids. Examples thereof are sodium chloride, sodium sulfate, sodium hydrogen sulfate, sodium carbonate, sodium hydrogen carbonate, sodium citrate, magnesium chloride, magnesium sulfate, magnesium carbonate, and magnesium hydrogen carbonate.

The agent according to the present invention is notable for the fact that oil-soluble ingredients accumulate predominantly in the hydrophobic phase (II) and therefore do not come into direct contact with the oxidizing agent-containing phase (I). This is particularly advantageous for stabilizing care-providing agents in the agent that have little stability in terms of oxidation. Such preferred care-providing substances are therefore oil-soluble care-providing substances, oil-soluble vitamins, and triglycerides, in particular vegetable ones and those that contain one or more unsaturated carbon-carbon bonds. In order to make the two-phase character visually apparent, it can likewise be useful if the hydrophobic phase (II) contains oil-soluble dyes.

A particular embodiment of the present invention is therefore characterized in that phase (II) of the agent according to the present invention additionally contains at least one predominantly oil-soluble care-providing component selected from oil-soluble dyes, oil-soluble care-providing substances, oil-soluble vitamins, and triglycerides, such that the quantitative and/or weight-based ratios of phase (I) to phase (II) that are recited above are not exceeded as a result of the presence of the additional oil-soluble care-providing components.

“Predominantly oil-soluble” refers, according to the present invention, to those compounds that have a water solubility of less than about 1 g per 1 L of water under standard conditions, but are readily soluble in nonpolar compounds (e.g. >10 g/kg of solution medium).

Oil-soluble care-providing substances are, for example, cosmetically effective terpenes and terpenoids such as, for example, bisabolol, and ubiquinones such as, for example, coenzyme Q-10.

Oil-soluble vitamins are in particular the compounds that are known by the collective terms vitamin A, vitamin D, vitamin E, and vitamin K. An agent preferred according to the present invention therefore contains at least one oil-soluble vitamin selected from vitamin A, vitamin D, vitamin E, and/or vitamin K, as well as vitamin P. “Vitamin A” encompasses retinoids, in particular all-trans-retinol. “Vitamin D,” also referred to as calciferols, encompasses 7,8-didehydrosterol derivatives, in particular the compounds referred to as cholecalciferol (vitamin D₃, calciol), ergocalciferol (vitamin D₂, ercalciol), 7,8-didehydrocholesterol (provitamin D₃, procalciol, procholecalciferol), and ergosterol (provitamin D₂). Further usable vitamin D analogs are calcidiol (25-hydroxycholecalciferol), calcitriol, hydroxycalcidiol, and vitamin D₁ (ergocalciferol and lumisterol). “Vitamin E” is the collective term for tocopherols, and encompasses in particular the chemical compounds α-tocopherol, β-tocopherol, γ-tocopherol, δ-tocopherol, and α-tocotrienol, β-tocotrienol, γ-tocotrienol, and δ-tocotrienol. “Vitamin K” is a collective term for various compounds having vitamin K activity, which derive from 2-methyl-1,4-naphthoquinone (vitamin K₃). Preferred representatives are vitamin K₁₍₂₀₎ (2-methyl-3-phytyl-1,4-naphthoquinone), phylloquinone (abbreviated: K),], vitamin K₂₍₃₅₎ (3-all-trans-farnesylgeranyl geranyl-2-methyl-1,4-naphtho quinone), vitamin K₃ (2-methyl-1,4-naphthoquinone, menadione, menaphthone), and the derived analogs vitamin K₄ (2-methyl-1,4-naphthalenediol), vitamin K₅ (4-amino-2-methyl-1-naphthol), vitamin K₆ (2-methyl-1,4-naphthalenediamine), and vitamin K₇ (4-amino-3-methyl-1-naphthol). “Vitamin P” is a collective term for rutins, in particular bioflavonoids such as troxerutin (vitamin P₄) and hesperidin.

“Triglycerides” is the collective term for esters of glycerol, which represent the principal constituents of natural oils. Triglycerides particularly preferred according to the present invention are those that contain at least one ester of an unsaturated fatty acid. Preferred unsaturated fatty acids are oleic acid, linoleic acid, and linolenic acid. Vegetable oils can also be used preferably as triglycerides, in particular those that have a positive influence on the hair surface. Particularly suitable triglycerides are, in particular, oils that are obtained from the seeds of Moring a pterygosperma (moring a oil), from the pits of Argania spinosa (argan oil), and/or from the seeds of Sclerocarya birrea (manila oil).

An embodiment of the first subject of the invention is therefore characterized in that the hydrophobic phase (II) additionally contains at least one oil that is selected from oils from the seeds of Moring a pterygosperma (moring a oil), from the pits of Argania spinosa (argan oil). and/or from the seeds of Sclerocarya birrea (marula oil).

The predominantly oil-soluble components are used preferably at a total weight from about 0.001 to about 5 wt %, in particular from about 0.01 to about 3 wt %, based in each case on the total weight of the hydrophobic phase (II).

The agents according to the present invention serve preferably to change the color of keratinic fibers. For this, the two-phase agent according to the present invention (M1) is mixed with a further agent (M2) containing at least one color-changing component, and the resulting ready-to-use preparation is placed onto the keratinic fibers.

The agents according to the present invention (M1) preferably have in this context, in particular in order to stabilize the oxidizing agent, an acidic pH in the range from about 2.5 to about 6. The “pH values” for purposes of the present invention are pH values that were measured at a temperature of 22° C.

Serving as color-changing components in the agent (M2) are, as lightening agents, preferably additional bleaching power intensifiers that intensify the action of the oxidizing agent from phase (I) of the two-phase agent according to the present invention, as well as color-imparting components.

In a preferred embodiment the agent (M2) therefore contains an additional bleaching power intensifier. Additional bleaching power intensifiers that can be used in the context of this invention are peroxo compounds, furthermore compounds that yield aliphatic peroxocarboxylic acids under perhydrolysis conditions and/or substituted perbenzoic acid, carbonic acid derivatives, alkylcarbonates or -carbamates, silylcarbonates and -carbamates.

The bleaching power intensifier is preferably selected from ammonium peroxodisulfate, alkali metal peroxodisulfates, ammonium peroxomonosulfate, alkali metal hydrogen peroxomonosulfates, alkali metal peroxodiphosphates, and alkaline earth metal peroxides. Particularly preferred bleaching power intensifiers are ammonium peroxodisulfate, potassium peroxodisulfate, sodium peroxodisulfate, potassium hydrogen peroxomonosulfate, potassium peroxodiphosphate, magnesium peroxide, and barium peroxide. Agents that contain as a bleaching power intensifier at least one inorganic salt selected from peroxomonosulfates and/or peroxodisulfates are particularly preferred. It has also proven to be particularly preferred if the agents (M2) contain at least two different peroxodisulfates. Preferred peroxodisulfate salts in this context are combinations of ammonium peroxodisulfate and potassium peroxodisulfate and/or sodium peroxodisulfate. The peroxo compounds are contained in a quantity from about 0.1 to about 25 wt %, in particularly in a quantity from about 0.5 to about 15 wt %, based on the total weight of the ready-to-use agent.

The persulfate salts resp. peroxodisulfate salts are as a rule used in anhydrous fashion and in the form of an optionally dedusted powder, a paste, or a pressed shaped element. The anhydrous agents (M2) can contain a further bleaching power intensifier instead of and/or in addition to the solid peroxo compounds.

Although in principle no limitations exist in terms of the formulation of the further agents (M2), it can be preferred according to the present invention if the agents (M2) are formulated to be anhydrous. “Anhydrous” for purposes of the present invention means a water content, based on the agent (M2), of less than 5 wt %, in particular less than 2 wt %. Hair-bleaching preparations that contain less than 0.1 wt % water can be preferred.

In a further, preferred embodiment, the agent (M2) can contain at least one cationic pyridinium derivative as a bleaching power intensifier. Preferred compounds are 4-acylpyridinium derivatives and 2-acylpyridinium derivatives. 2-Acetyl-1-methylpyridinium-p-toluenesulfonate and 4-acetyl-1-methylpyridinium-p-toluenesulfonate are particularly preferred in this context. Further preferred cationic pyridinium derivatives are cationic 3,4-dihydroisoquinolinium derivatives. N-methyl-3,4-dihydroisoquinolinium-p-toluenesulfonate is particularly preferred.

The bleaching power intensifiers used alongside or instead of peroxo compounds are contained in the agents (M2) preferably in quantities from about 0.05 to about 10 wt %, in particular in quantities from about 0.2 to about 5 wt %, based in each case on the total weight of the agent (M2).

In a further preferred embodiment the agent (M2) contains color-imparting components as a color-changing component. These can preferably be selected from at least one oxidation dye precursor and/or from at least one substantive dye.

Agents preferred according to the present invention for changing the color of keratinic fibers are characterized in that they contain at least one oxidization dye precursor. Agents that contain at least one oxidation dye precursor of the developer type (developer component), preferably in combination with at least one oxidation dye precursor of the coupler type (coupler component), are particularly preferred.

Preferred oxidation dye precursors of the developer type are p-phenylenediamine derivatives. Preferred p-phenylenediamines are selected from one or more compounds of the group that is constituted from p-phenylenediamine, p-toluoylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(2-hydroxyethyl)-p-phenylenediamine, 4-N,N-bis-(2-hydroxyethyl)amino-2-methylaniline, 4-N,N-bis-(2-hydroxyethyl)-amino-2-chloro aniline, 2-(2-hydroxyethyl)-p-phenylenediamine, 2-(1,2-dihydroxyethyl)-p-phenylenediamine, 2-fluoro-p-phenylenediamine, 2-isopropyl-p-phenylenediamine, N-(2-hydroxypropyl)-p-phenylenediamine, 2-hydroxymethyl-p-phenylenediamine, N,N-dimethyl-3-methyl-p-phenylenediamine, N-ethyl-N-2-hydroxyethyl-p-phenylenediamine, N-(2,3-dihydroxypropyl)-p-phenylenediamine, N-(4′-aminophenyl)-p-phenylenediamine, N-phenyl-p-phenylenediamine, 2-(2-hydroxyethyloxy)-p-phenylenediamine, 2-methoxymethyl-p-phenylenediamine, 2-(2-acetylaminoethyloxy)-p-phenylenediamine, N-(2-methoxyethyl)-p-phenylenediamine, N-(4-amino-3-methylphenyl)-N-[3-(1H-imidazol-1-yl)propyl]amine, 5,8-diaminobenzo-1,4-dioxan, and physiologically acceptable salts thereof. p-Phenylenediamine derivatives particularly preferred according to the present invention are selected from at least one compound of the group: p-phenylenediamine, p-toluoylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, 2-methoxymethyl-p-phenylenediamine, and physiologically acceptable salts thereof. It can furthermore be preferred according to the present invention to use as a developer component compounds that contain at least two aromatic nuclei that are substituted with amino and/or hydroxyl groups. Preferred binuclear developer components are selected in particular from at least one of the following compounds: N,N′-bis-(2-hydroxyethyl)-N,N-bis-(4′-aminophenyl)-1,3-diaminopropan-2-ol, N,N′-bis(2-hydroxyethyl)-N,N′-bis-(4′-aminophenyl)ethylenediamine, N,N′-bis-(4′-aminophenyl)tetramethylenediamine, N,N-bis-(2-hydroxyethyl)-N,N-bis-(4′-aminophenyl)tetramethylenediamine, N,N′-bis-(4-(methylamino)phenyl)tetramethylenediamine, N,N′-diethyl-N,N′-bis-(4′-amino-3′-methylphenyl)ethylenediamine, bis-(2-hydroxy-5-aminophenyl)methane, N,N-bis-(4′-aminophenyl)-1,4-diazacycloheptane, N,N-bis-(2-hydroxy-5-aminobenzyl)piperazine, N-(4′-aminophenyl)-p-phenylenediamine, and 1,10-bis-(2′,5′-diaminophenyl)-1,4,7,10-tetraoxadecane, as well as physiologically acceptable salts thereof. Very particularly preferred binuclear developer components are selected from among N,N-bis-(2-hydroxyethyl)-N,N-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, or a physiologically acceptable salt thereof. It can further be preferred according to the present invention to use as a developer component a p-aminophenol derivative or a physiologically acceptable salt thereof. Preferred p-aminophenols are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(2-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(2-hydroxyethylaminomethyl)phenol, 4-amino-2-(1,2-dihydroxyethyl)phenol, 4-amino-2-fluorophenol, 4-amino-2-chlorophenol, 4-amino-2,6-dichlorophenol, 4-amino-2-(diethylaminomethyl)phenol, as well as physiologically acceptable salts thereof. Very particularly preferred compounds are p-aminophenol, 4-amino-3-methylphenol, 4-amino-2-aminomethylphenol, 4-amino-2-(1,2-dihydroxyethyl)phenol, and 4-amino-2-(diethylaminomethyl)phenol. The developer component can further be selected from o-aminophenol and derivatives thereof, such as 2-amino-4-methylphenol, 2-amino-5-methylphenol, or 2-amino-4-chlorophenol. The developer component can furthermore be selected from heterocyclic developer components such as pyrimidine derivatives, pyrazole derivatives, pyrazolopyrimidine derivatives and pyrazolopyrazole derivatives, resp. physiologically acceptable salts thereof. Preferred pyrimidine derivatives are, in particular, the compounds 2,4,5,6-tetraminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine, and 2,5,6-triaminopyrimidine. Preferred pyrazole derivatives are, in particular, the compounds that are selected from among 4,5-diamino-1-methylpyrazole, 4,5-diamino-1-(2-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-t-butyl-1-methylpyrazole, 4,5-diamino-1-t-butyl-3-methylpyrazole, 4,5-diamino-1-(2-hydroxyethyl)-3-methylpyrazole, 4,5-diamino-1-ethyl-3-methylpyrazole, 4,5-diamino-1-ethyl-3-(4-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2-aminoethyl)amino-1,3-dimethylpyrazole, as well as physiologically acceptable salts thereof, but in particular 4,5-diamino-1-(2-hydroxyethyl)pyrazole. Preferred pyrazolopyrimidines are the compounds selected from among pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 3-amino-7-dimethylamino-2,5-dimethylpyrazolo[1,5-a]pyrimidine, as well as physiologically acceptable salts thereof and tautomeric forms thereof if a tautomeric equilibrium exists. A preferred pyrazolopyrazole derivative is 2,3-diamino-6,7-dihydro-1H,5H-pyrazolo[1,2-a]pyrazol-1-one.

Particularly preferred developer components are selected from at least one compound of the group that is constituted from p-phenylenediamine, p-toluoylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one, as well as physiologically acceptable salts thereof. Very particularly preferred developer components are p-toluoylenediamine, 2-(2-hydroxyethyl)-p-phenylenediamine, 2-methoxymethyl-p-phenylenediamine, N-(4-amino-3-methylphenyl)-N-[3-(1H-imidazol-1-yl)propyl]amine, and/or 4,5-diamino-1-(2-hydroxyethyl)pyrazole, as well as physiologically acceptable salts thereof.

The developer components are used preferably in a quantity from about 0.0001 to about 0.5 wt %, by preference about 0.001 to about 0.2 wt %, based in each case on the ready-to-use agent.

Coupler components alone do not produce any significant color in the context of oxidative coloring, but instead always require the presence of developer components. It is therefore preferred according to the present invention that when at least one coupler component is used, at least one developer component is additionally utilized. Coupler components for purposes of the invention allow at least one chemical residue of the coupler to be substituted with the oxidized form of the developer component, in which context a covalent bond forms between the coupler component and developer component.

Coupler components according to the present invention are preferably selected as at least one compound from one of the following classes: m-aminophenol, o-aminophenol, m-diaminobenzene, o-diaminobenzene, and/or derivatives thereof; naphthalene derivatives having at least one hydroxy group; di- resp. trihydroxybenzene; pyridine derivatives; pyrimidine derivatives; specific indole derivatives and indoline derivatives; pyrazolone derivatives (for example 1-phenyl-3-methylpyrazol-5-one); morpholine derivatives (for example 6-hydroxybenzomorpholine or 6-aminobenzomorpholine); quinoxaline derivatives (for example 6-methyl-1,2,3,4-tetrahydroquinoxaline), as well as mixtures of two or more compounds from one or more of those classes.

Preferred m-aminophenol coupler components are selected from at least one compound from the group that is constituted from 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-diethylaminophenol, N-cyclopentyl-3-aminophenol, 1,3-dihydroxy-5-(methylamino)benzene, 3-ethylamino-4-methylphenol, 2,4-dichloro-3-aminophenol, and physiologically acceptable salts thereof. Preferred m-diaminobenzene coupler components are selected from at least one compound from the group that is constituted from m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene, and physiologically acceptable salts thereof. Preferred o-diaminobenzene coupler components are selected from at least one compound from the group that is constituted from 3,4-diaminobenzoic acid and 2,3-diamino-1-methylbenzene and physiologically acceptable salts thereof. Preferred naphthalene derivatives having at least one hydroxy group are selected from at least one compound of the group that is constituted from 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene, and 2,3-dihydroxynaphthalene. Preferred di- resp. trihydroxybenzenes and derivatives thereof are selected from at least one compound of the group that is constituted from resorcinol, resorcinol monomethyl ether, 2-methylresorcinol, 5-methylresorcinol, 2,5-dimethylresorcinol, 2-chlororesorcinol, 4-chlororesorcinol, pyrogallol, and 1,2,4-trihydroxybenzene. Preferred pyridine derivatives are selected from at least one compound of the group that is constituted from 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 3,4-diaminopyridine, 2-(2-methoxyethyl)amino-3-amino-6-methoxypyridine, 2-(4′-methoxyphenyl)amino-3-aminopyridine, and physiologically acceptable salts thereof. Preferred pyrimidine derivatives are selected from at least one compound of the group that is constituted from 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, and physiologically acceptable salts thereof. Preferred indole derivatives are selected from at least one compound of the group that is constituted from 4-hydroxyindole, 6-hydroxyindole, and 7-hydroxyindole, and physiologically acceptable salts thereof. Preferred indoline derivatives are selected from at least one compound of the group that is constituted from 4-hydroxyindoline, 6-hydroxyindoline, and 7-hydroxyindoline, and physiologically acceptable salts thereof.

Coupler components particularly preferred according to the present invention are selected from among 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of those compounds or physiologically acceptable salts thereof. Resorcinol, 2-methylresorcinol, 5-amino-2-methylphenol, 3-aminophenol, 2-(2,4-diaminophenoxy)ethanol, 1,3-bis(2,4-diaminophenoxy)propane, 1-methoxy-2-amino-4-(2′-hydroxyethylamino)benzene, 2-amino-3-hydroxypyridine, and 1-naphthol, as well as a physiologically acceptable salt thereof, are very particularly preferred.

The coupler components are used preferably in a quantity from about 0.0001 to about 0.5 wt %, by preference about 0.001 to about 0.2 wt %, based in each case on the ready-to-use agent.

Developer components and coupler components are generally used in approximately molar quantities with respect to one another. Although molar utilization has proven useful, a certain excess of individual oxidation dye precursors is not disadvantageous, so that developer components and coupler components can exhibit a molar ratio from about 1 to about 0.5 to about 1 to about 3, in particular about 1 to about 1 to about 1 to about 2.

In addition, the color-changing agents can contain at least one substantive dye. These are dyes that absorb directly onto the hair and do not require an oxidative process for the formation of color. Substantive dyes can be subdivided into anionic, cationic, and nonionic substantive dyes. They are usually nitrophenylenediamines, nitroaminophenols, azo dyes, anthraquinones, or indophenols. Substantive dyes are used in each case preferably in a quantity from about 0.0001 to about 0.2 wt %, preferably from about 0.001 to about 0.1 wt %, based in each case on the total utilization preparation. The total quantity of substantive dyes is by preference at most about 0.1 wt %.

Preferred anionic substantive dyes are the compounds known under the international designations resp. commercial names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue, and tetrabromophenol blue. Preferred cationic substantive dyes are cationic triphenylmethane dyes such as, for example, Basic Blue 7, Basic Blue 26, Basic Violet 2, and Basic Violet 14, aromatic systems that are substituted with a quaternary nitrogen group, for example Basic Yellow 57, Basic Red 76, Basic Blue 99, HC Blue 16 (Bluequat B), Basic Brown 16, and Basic Brown 17, as well as substantive dyes which contain a heterocycle that comprises at least one quaternary nitrogen atom, in particular Basic Yellow 87, Basic Orange 31, and Basic Red 51. The cationic substantive dyes that are marketed under the Arianor® trademark are likewise particularly preferred cationic substantive dyes according to the present invention. Nonionic nitro and quinone dyes, and neutral azo dyes, are particularly suitable as nonionic substantive dyes. Preferred nonionic substantive dyes are the compounds known under the international designations resp. commercial names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol. Dye combinations preferred according to the present invention are those that contain at least the combination of tetrabromophenol blue and Acid Red 92; tetrabromophenol blue and Acid Red 98; tetrabromophenol blue and Acid Red 94; tetrabromophenol blue and Acid Red 87, or tetrabromophenol blue and Acid Red 51.

Ready-to-use agents according to the present invention are by preference aqueous, flowable preparations. The agents according to the present invention can furthermore contain all active substances, additives, and adjuvants known for such preparations.

The oxidizing agent preparation can also be applied onto the hair together with a catalyst that activates oxidation of the dye precursors, e.g. by atmospheric oxygen. Such catalysts are, for example, specific enzymes, iodides, quinones, or metal ions. Enzymes suitable for this are, for example, peroxidases, which can considerably intensify the action of small quantities of hydrogen peroxide. A use of specific metal ions or metal complexes can also be preferred. Suitable metal ions are, for example, Zn²⁺, Cu²⁺, Fe²⁺, Fe³⁺, Mn²⁺, Mn⁴⁺, Li⁺, Mg²⁺, Ca²⁺, Ce⁴⁺, V³⁺, Co²⁺, Ru³⁺, and Al³⁺. Zn²⁺, Cu²⁺, and Mn²⁺ are particularly preferred in this context.

It has furthermore proven to be advantageous if the oxidizing agent preparations contain at least one stabilizer or complexing agent. Particularly preferred stabilizers are phenacetin, alkali benzoates (sodium benzoate), and salicylic acid.

The use of so-called complexing agents is also preferred according to the present invention. Complexing agents are substances that can complex metal ions. Preferred complexing agents are so-called chelate complexing agents, i.e. substances that form cyclic compounds with metal ions, where an individual ligand occupies more than one coordination site on a central atom, i.e. is at least “double-toothed.” Usual chelate complexing agents that are preferred in the context of the present invention are, for example, polyoxycarboxylic acids, polyamines, ethylenediaminetetraacetic acid (EDTA), nitrilotriacetic acid (NTA), and hydroxyethanediphosphonic acids resp. alkali salts thereof. Also usable according to the present invention are complexing polymers, i.e. polymers that carry either in the main chain itself, or laterally thereto, functional groups that can act as ligands and react with suitable metal atoms, usually accompanied by the formation of chelate complexes. The polymer-bound ligands of the resulting metal complexes can derive from only one macromolecule or else can belong to different polymer chains. Complexing agents preferred according to the present invention are nitrogen-containing polycarboxylic acids, in particular EDTA, and phosphonates, by preference hydroxyalkane- resp. aminoalkanephosphonates, and in particular 1-hydroxyethane-1,1-diphosphonate (HEDP) resp. the di- or tetrasodium salt thereof, and/or ethylenediaminetetramethylenephosphonate (EDTMP) resp. the hexasodium salt thereof, and/or diethylenetriaminepentamethylenephosphonate (DTPMP) resp. the hepta- or octasodium salt thereof.

Further active substances, adjuvants, and additives usable according to the present invention are, for example, nonionic polymers (such as vinylpyrrolidinone/vinyl acrylate copolymers, polyvinylpyrrolidinone, vinylpyrrolidinone/vinyl acetate copolymers, and polysiloxanes); zwitterionic and amphoteric polymers (such as acrylamidopropyltrimethylammonium chloride/acrylate copolymers and octylacrylamide/methyl methacrylate/tert-butylaminoethyl methacrylate/2-hydroxypropyl methacrylate copolymers); thickening agents (such as agar-agar, guar gum, alginates, xanthan gum, gum arabic, karaya gum, locust bean flour, linseed gums, dextrans, cellulose derivatives, e.g. methyl cellulose, hydroxyalkyl cellulose, and carboxymethyl cellulose, starch fractions and derivatives such as amylose, amylopectin, and dextrins, clays such as bentonite, or entirely synthetic hydrocolloids such as, for example, polyvinyl alcohol); structuring agents (such as sugars, maleic acid, and lactic acid) and consistency agents (such as sugar esters, polyol esters, or polyolalkyl ethers); protein hydrolysates (in particular hydrolysates of elastin, collagen, keratin, milk protein, soy protein, and wheat protein, condensation products thereof with fatty acids); perfume oils; cyclodextrins; solvents and solubilizers (such as ethanol, isopropanol, ethylene glycol, propylene glycol, glycerol, dimethyl isosorbide, and diethylene glycol); defoamers such as silicones; dyes and pigments for coloring the agent; anti-dandruff active substances (such as piroctone olamine, zinc omadine, and climbazol); light-protection agents (in particular derivatized benzophenones, cinnamic acid derivatives, and triazines); active substances (such as allantoin, pyrrolidonecarboxylic acids, cholesterol, and salts thereof; further fats and waxes (such as fatty alcohols, beeswax, montan wax, and paraffins); swelling and penetration substances (such as glycerol, propylene glycol monoethyl ether, carbonates, hydrogen carbonates, guanidines, ureas, as well as primary, secondary, and tertiary phosphates); opacifiers (such as latex, styrene/PVP and styrene/acrylamide copolymers); luster agents (such as ethylene glycol mono- and distearate as well as PEG-3 distearate); propellants (such as propane/butane mixtures, N₂O, dimethyl ether, CO₂, and air); and antioxidants.

One skilled in the art will arrive at a selection of these further substances in accordance with the desired properties of the agents. With regard to further optional components, as well as the quantities of those components used, reference is made expressly to the relevant manuals known to one skilled in the art, e.g. Kh. Schrader, Grundlagen and Rezepturen der Kosmetika [Cosmetics fundamentals and formulations], 2nd ed., Hiithig Buch Verlag, Heidelberg, 1989.

Preferred ready-to-use agents made up of a two-phase agent according to the present invention (M1) and a color-changing agent (M2) preferably have a pH in the range from about 6 to about 12. Particularly preferred agents are characterized in that they have an alkaline pH. A further preferred embodiment of the present agent consists in the fact that the ready-to-use agent has a pH between about 7.0 and about 12.0, preferably between about 8.0 and about 11.0. The pH values for purposes of the present invention are pH values that were measured at a temperature of 22° C.

The pH is usually adjusted using pH adjusting agents. One skilled in the art of cosmetics is familiar, for purposes of adjusting the pH, with acidifying and alkalizing agents usual in cosmetics. The alkalizing agents usable for adjusting the pH are typically selected from inorganic salts, in particular of the alkali and alkaline-earth metals, organic alkalizing agents, in particular amines, basic amino acids and alkanolamines, and ammonia. Acidifying agents preferred according to the present invention are edible acids such as, for example, citric acid, acetic acid, malic acid, or tartaric acid, as well as dilute mineral acids.

Organic alkalizing agents usable according to the present invention are preferably selected from alkanolamines of primary, secondary, or tertiary amines with a C₂ to C₆ alkyl base element that carries at least one hydroxyl group. Alkanolamines particularly preferred according to the present invention are selected from the group: 2-aminoethan-1-ol (monoethanolamine), 2-amino-2-methylpropan-1-ol, and 2-amino-2-methylpropane-1,3-diol). A particularly preferred alkanolamine is monoethanolamine. Suitable basic amino acids are lysine, arginine, and ornithine. The inorganic alkalizing agents according to the present invention are preferably selected form the group that is constituted from sodium hydroxide, potassium hydroxide, calcium hydroxide, barium hydroxide, sodium phosphate, potassium phosphate, sodium silicate, potassium silicate, sodium carbonate, and potassium carbonate.

Utilization temperatures can be in a range between about 15 and about 40° C. After a contact time from about 2 to about 60, preferably about 5 to about 45 minutes, the hair-bleaching agent is removed from the hair by being rinsed out. Subsequent washing with a shampoo is superfluous if a highly surfactant-rich carrier was used.

It is important that the agents (M1) and (M2) be sufficiently liquid that good mixing with one another is possible. It is advantageous for this purpose if the two agents are not present as a paste, viscous cream, or thickened gel, but instead have sufficient flowability. In addition, the ready-to-use agents must possess, after mixing of the individual components, rheological properties that permit application onto the fibers to be colored but at the same time prevent the agent from running or flowing out from the site of action during the utilization time. The utilization mixtures therefore preferably possess a viscosity from about 5 to about 100 Pa·s, preferably from about 10 to about 50 Pa·s, in particular from about 10 to about 20 Pa·s, and particularly preferably from about 10 to about 16 Pa·s (Brookfield, 22° C., spindle 5, 4 rpm).

Depending on the composition of the agents (M1) and (M2), it is preferred to produce the utilization mixtures only immediately before utilization, by mixing the agent (M1) and the agent (M2). This can be advantageous in particular in the context of incompatibilities between individual ingredients. A preferred administration form of the ready-to-use agent is therefore a separate packaging unit in which agents (M1) and (M2) are each present packaged separately from one another.

A further subject of the present invention is therefore a multi-component packaging unit (kit of parts) that contains at least two containers formulated separately from one another, where a first container (C1) contains a cosmetic agent (M1) according to the first subject of the invention, and a second container (C2) contains a color-changing preparation (M2), where the color-changing preparation (M2) contains, in a cosmetic carrier, at least one color-changing component, preferably at least one oxidation dye precursor.

A “container” is understood in the context of the present invention as a casing that exists in the form of an optionally reclosable bottle, a tube, a can, a pouch, a sachet, or similar casings. No limits are imposed on the casing material according to the present invention. The casings are, however, preferably made of glass or plastic. An embodiment in which the casing of the container that contains the agent (M1) is transparent to the user is particularly preferred for visualization of the two-phase agent (M1). A preferred embodiment of the multi-component packaging unit according to the present invention is therefore characterized in that the first container (C1), containing the agent (M1), has a transparent package, preferably a transparent plastic package.

It can furthermore be advantageous according to the present invention if the aforesaid kit of parts contains at least one further hair treatment agent in a separate container, in particular a conditioning agent. The packaging unit can moreover encompass application aids, such as combs, hairbrushes, or brushes, personal protective apparel, in particular disposable gloves, and optionally a user manual.

The statements made above regarding the previous subjects of the invention apply analogously, mutatis mutandis, in terms of the preferred embodiments of the agents (M1) and (M2).

In the context of utilization of the multi-component packaging unit, it can be immaterial whether firstly the two phases of the agent (M1) are briefly blended by vigorous shaking and combined with the agent (M2), before the phases separate again, in order to furnish the ready-to-use color-changing preparation; or if firstly the agent (M2) is combined with the agent (M1) and the ready-to-use mixture is then produced by thorough mixing.

A further subject of the invention is therefore a method for changing the color of keratinic fibers, in particular human hair, which is characterized in that from a multi-component packaging unit according to the preceding subject of the invention, the two agents (M1) and (M2) are combined in one of the containers (C2) or (C1), the reclosed container is thereupon shaken, and the ready-to-use color-changing agent resulting in the container is then applied onto the fibers, left on the fibers for a contact time from 5 to 60 minutes, and lastly rinsed out.

For improved blending, it is advantageous if the container (C1) that contains the two-phase agent (M1) possesses a reclosable opening, for example a snap closure or screw closure. This enables easier addition of the color-changing agent from the container (C2), which in turn exists preferably in the form of a pouch or sachet in the case of anhydrous, in particular powdered color-changing agents, or in the form of a tube in the case of flowable color-changing agents. It is preferred to mix the individual preparations and to apply the ready-to-use agent quasi-synchronously onto the keratinic fibers.

A further particularly preferred embodiment of this subject of the invention is therefore a method for changing the color of keratinic fibers, in particular human hair, which is characterized in that from a multi-component packaging unit in accordance with the previous subject of the invention, the contents of the container (C2) are introduced into the container (C1), the reclosed container (C1) is thereupon shaken, and the ready-to-use color-changing agent resulting in the container (C1) is then applied onto the fibers, left on the fibers for a contact period from about 5 to about 60 minutes, and then rinsed out.

Lastly, a further subject of the invention is a method for changing the color of keratinic fibers, in particular human hair, which is characterized in that from a multi-component packaging unit in accordance with the previous subject of the invention, the container (C1) is shaken, the resulting mixtures of phases (I) and (II) is immediately thereafter thoroughly mixed with a coloring preparation of the container (C2), the resulting ready-to-use color-changing agent is then applied onto the fibers, left on the fibers for a contact period from about 5 to about 60 minutes, and lastly rinsed out.

In the case of a color-imparting agent, the preferred contact time is about 5 to about 40 minutes, preferably about 10 to about 30 minutes. In the case of lightening or bleaching color-changing agents, the preferred contact time is about 30 to about 60 minutes, preferably about 40 to about 60 minutes.

The statements made above apply analogously, mutatis mutandis, in the context of this subject of the invention. The Examples below are intended to explain the subject matter of the present invention in more detail without in any way limiting it.

### EXAMPLES

1) Two-Phase Developer Preparations (Table 1; Quantities Indicated in Wt %)

The following two-phase developer preparations were produced using different hydrophobic oil mixtures.

TABLE 1

| Raw materials: | 1 | 2 | 3 |
|---|---|---|---|
| Aqueous phase: | | | |
| Sodium hydroxide, 45% techn. | 0.76 | 0.76 | 0.76 |
| Dipicolinic acid | 0.10 | 0.10 | 0.10 |
| Disodium pyrophosphate | 0.03 | 0.03 | 0.03 |
| HEDP, aqueous, 60% | 1.35 | 1.35 | 1.35 |
| Sodium laureth sulfate (2EO), 27% | 1.80 | 1.80 | 1.80 |
| Aculyn ® 33A | 15.50 | 15.50 | 15.50 |
| Hydrogen peroxide, aqueous, 50% | 12.00 | 12.00 | 12.00 |
| Hydrophobic oil phase: | | | |
| Isopropyl palmitate | 4.60 | 4.60 | 4.60 |
| Controx ® KS C | 0.07 | 0.07 | 0.07 |
| Isopropyl isostearate | 4.90 | — | — |
| Eutanol ® G16S | — | 4.90 | — |
| 2-Ethylhexyl palmitate | — | — | 4.90 |
| Marula oil | 0.10 | 0.10 | 0.10 |

| | |
|---|---|
| Water, deionized | to 100 |

Raw materials: Aculyn ® 33A (approx. 28%; INCI name: Acrylates Copolymer, Aqua; Rohm & Haas); Controx KS C (INCI name: Tocopherol, Hydrogenated Palm Glycerides Citrate; BASF); Eutanol G16S (INCI name: Hexyldecyl Stearate; BASF).

In order to manufacture the developer preparations, the raw materials with the exception of the oils (isopropyl palmitate, Controx KS C, isopropyl stearate, marula oil (1); isopropyl palmitate, Controx KS C, Eutanol G16S, marula oil (2); and isopropyl palmitate, Controx KS C, 2-ethylhexyl palmitate, manila oil (3)) were premixed. The respective hydrophobic, clear oil phase was then added.

The aqueous phase and the hydrophobic oil phase are present separately from one another when undisturbed. They can be mixed outstandingly well by shaking. Storage of the two-phase agent presented no problems at all. No changes were identified even in a context of temperature fluctuations and storage at lower temperatures (<15° C.).

2) Color Creams I to III (Table 2; Quantities Indicated in Wt %)

| Raw materials | I | II | III |
|---|---|---|---|
| Lanette ® D | 6.60 | 6.60 | 6.60 |
| Lorol ® C12-18 techn. | 2.40 | 2.40 | 2.40 |
| Eumulgin ® B 2 | 0.60 | 0.60 | 0.60 |
| Eumulgin ® B 1 | 0.60 | 0.60 | 0.60 |
| Lamesoft ® PO 65 | 2.00 | 2.00 | 2.00 |
| Akypo Soft ® 45HP | 10.00 | 10.00 | 10.00 |
| Texapon ®K 14 S Special, 70% | 2.80 | 2.80 | 2.80 |
| Product W 37194 ® | 3.75 | 3.75 | 3.75 |
| p-Toluylenediamine sulfate | 0.37 | 2.21 | 1.53 |
| 2,4,5,6-Tetraaminopyrimidine sulfate | 1.46 | | |
| Resorcinol | 0.10 | 0.79 | 0.41 |
| 2-Methylresorcinol | 0.90 | | 0.34 |
| m-Aminophenol | | 0.29 | |
| Lehmann's Blue | | 0.05 | |
| 2-Amino-3-hydroxypyridine | | | 0.05 |
| Ammonium sulfate | 0.02 | | |
| Sodium sulfite, anhydrous | 0.40 | 0.40 | 0.40 |
| Ascorbic acid | 0.10 | 0.10 | |
| Sodium hydroxide, aqueous, 45% | 1.90 | 2.00 | 1.50 |
| Sodium silicate 40/42 | 0.50 | 0.50 | 0.50 |
| L-Serine | 1.00 | 1.00 | 1.00 |
| Ajidew ® NL50 | | | 1.00 |
| Turpinal ® SL | 0.20 | 0.20 | 0.20 |
| Perfume | 0.30 | 0.30 | 0.30 |
| Water, deionized | to 100 | to 100 | to 100 |

Raw materials: Lanette D (INCI name: Cetearyl Alcohol; BASF); Lorol C12-18 techn. (INCI name: Coconut Alcohol; BASF); Eumulgin B 2 (INCI name: Ceteareth-20; BASF); Eumulgin B 1 (INCI name: Ceteareth-12; BASF); Akypo Soft 45HP (approx. 21%, INCI name: Sodium Laureth-6 Carboxylate, Aqua; KAO); Texapon K 14 S Special (ca. 70%, INCI name: Sodium Myreth Sulfate, Aqua; BASF); Product W 37194 (ca. 20%, INCI name: Acrylamidopropyltrimonium Chloride/Acrylates Copolymer, Aqua; Stockhausen); Ajidew NL 50 (INCI name: Sodium PCA; Ajinomoto); Turpinal ® SL (INCI name: Etidronic Acid, Aqua; Thermphos).

The fat base was melted respectively at 80° C. and dispersed with a portion of the quantity of water. The remaining formulation constituents were then incorporated successively while stirring. The volume was brought up to 100 wt % with water, and formulations I to III were stirred until cold.

The respective color cream had one of the developer solutions added to it at room temperature at a weight ratio of 1:1, and was thoroughly mixed.

1. A cosmetic agent for treating keratinic fibers, the cosmetic agent having at least two phases present alongside one another but separated from one another by a phase boundary,
where a first phase (I) represents an aqueous phase that contains at least one chemical oxidizing agent, and
where a second phase (II) represents a hydrophobic oil phase that contains a mixture of isopropyl palmitate (II-1) and at least one further liquid, branched carboxylic acid ester (II-2).

2. The cosmetic agent according to claim 1, wherein the at least one chemical oxidizing agent of the first phase (I) is selected from hydrogen peroxide and/or from one of its solid addition products with inorganic and/or organic compounds.

3. The cosmetic agent according to claim 1, wherein a weight ratio of isopropyl palmitate (II-1) to a sum of all further carboxylic acid esters (II-2) in phase (II) is about 20:80 to about 80:20.

4. The cosmetic agent according to claim 1, wherein the at least one further liquid, branched carboxylic acid ester(s) of phase (II) is/are selected from esters of straight-chain or branched C₂ to C₃₀ carboxylic acids with branched C₃ to C₃₀ alcohols.

5. The cosmetic agent according to claim 4, wherein the at least one further liquid, branched carboxylic acid ester(s) of phase (II) is/are selected from isopropyl isostearate, 2-ethylhexyl palmitate, hexyldecyl palmitate, hexyldecyl stearate, isopropyl stearate, hexyldecyl laurate, isodecyl neopentanoate, isononyl isononanoate, 2-ethylhexyl stearate, isopropyl oleate, isooctyl stearate, isononyl stearate, isocetyl stearate, isononyl isononanoate, isotridecyl isononanoate, cetearyl isononanoate, propylheptyl caprylate, cetearyl isononanoate, 2-ethylhexyl laurate, 2-ethylhexyl isostearate, 2-ethylhexyl cocoate, 2-octyldodecyl palmitate, butyl octanoic acid 2-butyl octanoate, and/or diisotridecyl acetate.

6. The cosmetic agent according to claim 1, the cosmetic agent comprising, based on its total weight, about 3 to about 30 wt % of the hydrophobic oil phase (II).

7. The cosmetic agent according to claim 1, wherein the aqueous phase (I) further contains at least one anionic polymeric thickening agent that is selected from crosslinked or uncrosslinked copolymers that contain at least two different monomers from the group of acrylic acid, methacrylic acid, C₁ to C₆ alkyl esters of acrylic acid, and/or C₁ to C₆ alkyl esters of methacrylic acid.

8. The cosmetic agent according to claim 1, wherein the cosmetic agent comprises nonionic, anionic, zwitterionic, and/or amphoteric surfactants and/or emulsifier agents at a total weight of less than about 5 wt %, based on the total weight of the cosmetic agent.

9. A multi-component packaging unit (kit of parts) containing at least two containers formulated separately from one another, where a first container (C1) contains a cosmetic preparation (M1) for treating keratinic fibers, the cosmetic preparation (M1) having at least two phases present alongside one another but separated from one another by a phase boundary, where a first phase (I) represents an aqueous phase that contains at least one chemical oxidizing agent, and where a second phase (II) represents a hydrophobic oil phase that contains a mixture of isopropyl palmitate (II-1) and at least one further liquid, branched carboxylic acid ester (II-2), and a second container (C2) contains a color-changing preparation (M2), where the color-changing preparation (M2) contains, in a cosmetic carrier, at least one color-changing component.

10. The multi-component packaging unit of claim 9, wherein the at least one color-changing component comprises at least one oxidation dye precursor.

11. A method for changing the color of keratinic fibers, in particular human hair, the method comprising the steps of:
providing a multi-component packaging unit containing at least two containers formulated separately from one another, where a first container (C1) contains a cosmetic preparation (M1) for treating keratinic fibers, the cosmetic preparation having at least two phases present alongside one another but separated from one another by a phase boundary, where a first phase (I) represents an aqueous phase that contains at least one chemical oxidizing agent, and where a second phase (II) represents a hydrophobic oil phase that contains a mixture of isopropyl palmitate (II-1) and at least one further liquid, branched carboxylic acid ester (II-2), and a second container (C2) contains a color-changing preparation (M2), where the color-changing preparation (M2) contains, in a cosmetic carrier, at least one color-changing component;
combining the two preparations (M1) and (M2) in one of the containers (C2) or (C1) and closing the container;
shaking the closed container to form a ready-to-use color-changing agent;
applying the ready-to-use color-changing agent resulting in the container onto the fibers and leaving the ready-to-use color-changing agent on the fibers for a contact time from about 5 to about 60 minutes; and
rinsing the ready-to-use color-changing agent out of the fibers.
